(19) United States
(12) Patent Application Publication
(10) Pub. No.: US 2021/0100952 A1
(43) Pub. Date: Apr. 8, 2021

(54) Systems and Methods for Treating Hypoglycemia

(71) Applicant: Sean Brown, Phoenix, AZ (US)

(72) Inventor: Sean Brown, Phoenix, AZ (US)

(21) Appl. No.: 16/851,774

(22) Filed: Apr. 17, 2020

Related U.S. Application Data
(60) Provisional application No. 62/911,206, filed on Oct. 4, 2019.

(51) Int. Cl. A61M 5/172

(52) U.S. Cl. CPC A61M 5/1723; A61M 2230/201; A61M 2205/50; A61M 2205/3303; A61M 2230/10

(57) ABSTRACT

Systems and methods for treating hypoglycemia generally comprise a processor and a medicament injection unit. A processor may receive continuous glucose sensor data indicative of a host's blood glucose concentration. The processor may determine whether a host's blood glucose concentration is below a predetermined threshold setting. Based at least in part on the host's blood glucose concentration, the medicament injection unit may be configured to administer a medicament to the host.

### CROSS REFERENCE TO RELATED APPLICATIONS

This application claims the benefit of U.S. Provisional Patent Application No. 62/911,206, filed Oct. 4, 2019 and incorporates the disclosure of each application by reference.

### BACKGROUND

### 1. Field of the Invention

Aspects of this document relate generally to systems and methods for autonomously treating severe hypoglycemia.

### 2. Description of Related Art

Diabetes mellitus is a disorder characterized by chronic hyperglycemia. There are two main types; type 1 (formerly known as insulin dependent) and type 2 (formerly known as non-insulin dependent. Due to a variety of factors, persons with diabetes mellitus (diabetics) may suffer hypoglycemia. Hypoglycemia is a condition where a diabetic's blood sugar (glucose) concentration is lower than normal. If a diabetic's glucose concentration becomes too low (hypoglycemia), the victim may experience complications such as, but not limited to, fatigue, irregular or fast heartbeat, confusion, abnormal behavior, visual disturbances, and as it worsens towards severe hypoglycemia (an event requiring the assistance of another person to actively administer carbohydrate, glucagon, or other resuscitative actions), seizures, loss of consciousness, coma or death.

Diabetics may use continuous glucose monitoring devices to monitor their glucose levels. Newer versions of these devices can provide blood glucose data in real time and their use has reduced the frequency but not eliminated the incidence of severe hypoglycemic events in diabetics. For example, diabetics without using continuous glucose monitoring devices experience on average more than two severe hypoglycemic events per year. After consistently using a continuous glucose monitoring device, diabetics trend towards experiencing around one severe hypoglycemic event per year.

Diabetics experiencing severe hypoglycemia must treat it by increasing their glucose concentration. Currently, a standard treatment for severe hypoglycemia is an intramuscular dose of glucagon (a hormone that raises blood sugar levels). Currently, glucagon preparations/kits are delivered by another person, different from the person experiencing severe hypoglycemia. Delivery of glucagon in severe hypoglycemia can be problematic if there is not another person present to recognize the severe hypoglycemia and deliver the glucagon. CGMs provide blood glucose concentration levels, but currently lack the automated counter-regulatory capacity to prevent or immediately treat severe hypoglycemia. As such, there exists an unmet need to unite existent CGM technology with the delivery of standard glucagon treatment in an automated and on-site fashion.

So as to reduce the complexity and length of the Detailed Specification, and to fully establish the state of the art in certain areas of technology, Applicant(s) herein expressly incorporate(s) by reference all of the following materials identified in each numbered paragraph below.

U.S. Pat. No. 10,403,012 of Kamath et al. describes an integrated medicament delivery device for use with continuous analyte sensor. An integrated system for the monitoring and treating diabetes is provided, including an integrated receiver/hand-held medicament injection pen, including electronics, for use with a continuous glucose sensor. In some embodiments, the receiver is configured to receive continuous glucose sensor data, to calculate a medicament therapy (e.g., via the integrated system electronics) and to automatically set a bolus dose of the integrated hand-held medicament injection pen, whereby the user can manually inject the bolus dose of medicament into the host. In some embodiments, the integrated receiver and hand-held medicament injection pen are integrally formed, while in other embodiments they are detachably connected and communicated via mutually engaging electrical contacts and/or via wireless communication.

U.S. Pat. No. 10,406,286 of Hayter describes a closed loop control with reference measurement and methods thereof. Methods, system and devices for monitoring a plurality of parameters associated with a closed loop control operation including continuously monitoring a physiological condition and automatic administration of a medication, detecting an impending administration of the medication level exceeding a predetermined threshold level, retrieving one or more signals associated with the monitored physiological condition, determining an accuracy indication of the retrieved one or more signals associated with the monitored physiological condition, and executing the administration of the medication level based on the accuracy indication are provided.

U.S. Pat. No. 10,383,998 of Allerdings describes a method and monitoring device for monitoring operation of a drug delivery device. The present invention relates to a method and to a monitoring device for monitoring operation of a drug delivery device, the monitoring device comprising of at least a first and a second sensor arranged at a distance from each other with regard to a first direction and being adapted to generate a first and a second electrical signal in response to an operation of the device, a processing unit configured to determine a time delay between the first and the second electrical signals and being adapted to determine at least one state parameter of the drug delivery device on the basis of said time delay.

U.S. Pat. No. 10,391,252 of Haupt describes a supplemental device for attachment to an injection device. A supplemental device for attachment to an injection device, the supplemental device comprising: a housing; an electromechanical switch arrangement having an open state and a closed state, the electromechanical switch arrangement comprising a protrusion configured to contact a surface of the injection device while the supplemental device is attached to the injection device; a dose dialed detector operable to detect a dose of medicament dialed into the attached injection device; and a processor arrangement configured to: monitor the detected dose of medicament dialed into the attached injection device; detect a change in the state of the electromechanical switch arrangement; and change a display output of the supplemental device when a change in the state of the electromechanical switch arrangement is detected while a zero dose is dialed into the attached injection device.

U.S. Pat. No. 10,363,365 of Bazargan describes infusion devices and related consumable calibration methods Infusion systems including infusion devices and consumables and related operating methods are provided. An exemplary method of operating an infusion device operable to deliver a fluid influencing a physiological condition to a body of a user involves a control module of the infusion device obtaining calibration data associated with a consumable coupled to the infusion device via an interface of the infusion device, determining a delivery command for delivering the fluid to the body of the user based at least in part on the calibration data, and operating a pumping mechanism to deliver the fluid from a reservoir of the consumable to the body of the user in accordance with the delivery command.

Publication Lykke et al. 2016. Hypoglycemia-Induced Changes in the Electroencephalogram: An Overview. J. of Diabetes Science and Tech 10(6): 1259-1267.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), Applicant(s) will amend the specification to expressly recite the essential material that is incorporated by reference as allowed by the applicable rules.

### SUMMARY

The present invention provides among other things, systems and method for treating hypoglycemia. Systems and methods for treating hypoglycemia according to the various aspects of the present invention may comprise a processor and a medicament injection unit. A processor may receive continuous glucose sensor data indicative of a host's blood glucose concentration. The processor may determine whether a host's blood glucose concentration is below a predetermined threshold setting. Based at least in part on the host's blood glucose concentration, the medicament injection unit may be configured to administer a medicament to the host.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographers, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of pre-AIA 35 U.S.C. § 112, ¶6 and post-AIA 35 U.S.C. § 112(f). Thus, the use of the words “function,” “means” or “step” in the Detailed Description or Description of the Drawings or claims is not intended to somehow indicate a desire to invoke the special provisions of pre-AIA 35 U.S.C. § 112, ¶6 or post-AIA 35 U.S.C. § 112(f), to define the invention. To the contrary, if the provisions of pre-AIA 35 U.S.C. § 112, ¶6 or post-AIA 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of pre-AIA 35 U.S.C. § 112, ¶6 or post-AIA 35 U.S.C. § 112(f). Moreover, even if the provisions of pre-AIA 35 U.S.C. § 112, ¶6 or post-AIA 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE SEVERAL VIEWS OF DRAWINGS

A more complete understanding of the present invention may be derived by referring to the detailed description when considered in connection with the following illustrative figures. In the figures, like reference numbers refer to like elements or acts throughout the figures.

FIG. 1 depicts one embodiment of the patch pump system coupled to a continuous glucose monitor sensor, host, controller, and electroencephalogram sensor.

FIG. 2 depicts one embodiment of the patch pump operating after receiving input from a continuous glucose monitor sensor, controller, and electroencephalogram sensor.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

In one application, representative implementations of the present invention may be applied to any system for treating and detecting when a host is experiencing changes in blood glucose concentration indicating a need for medicament. For example, having blood glucose concentrations less than 54 mg/dL may indicate a need for medicament administration to the host. Certain representative implementations of the patch pump may include, for example: surgical implantation within the host, a wearable upon the host, or other placement such that a portion of the patch pump is capable of administering a medicament to the host. Generally, the patch pump is configured to receive physiologic signs from a host and depending on those physiologic signs, administer a medicament to a host who is experiencing blood glucose concentration changes that indicate a need for medicament. The patch pump may operate in conjunction with any system that may help detect whether a host is experiencing changes in blood glucose concentration indicating a need for medicament.

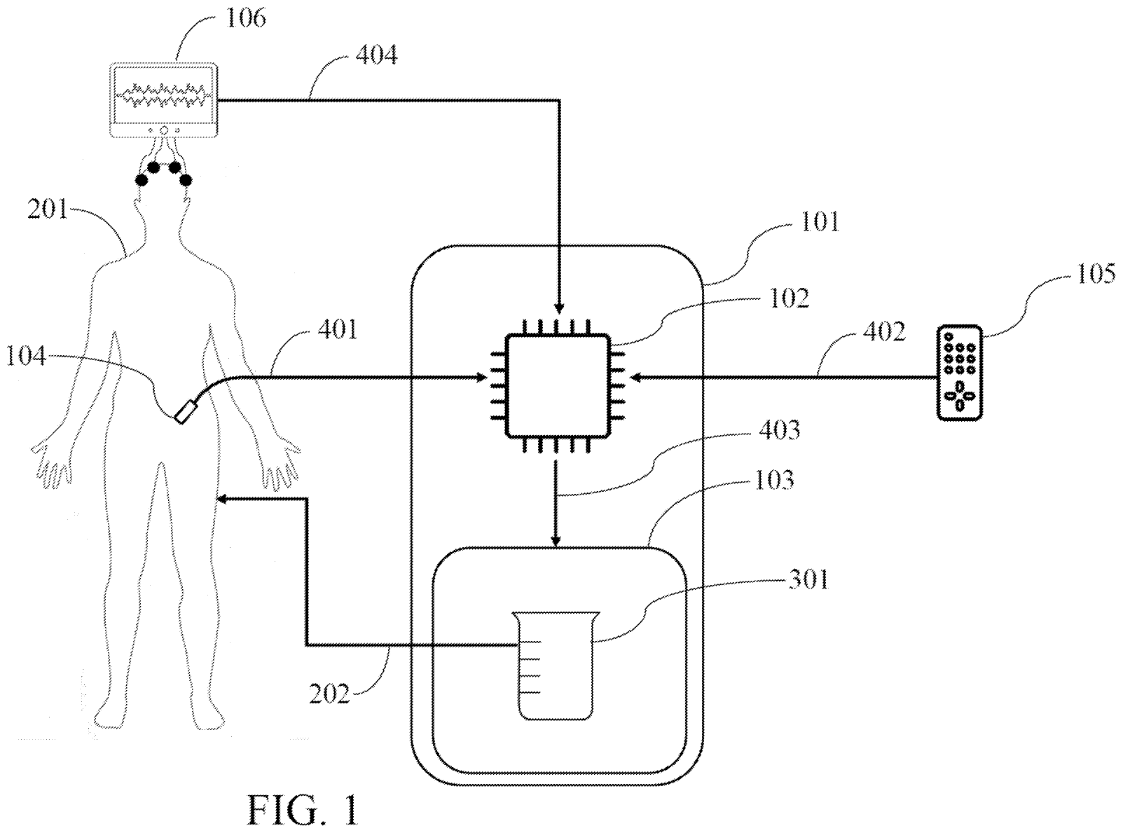

For example, referring to FIG. 1, a patch pump 101 according to various aspects of the present invention comprises a processor 102 and a medicament injection unit 103. In one exemplary embodiment, the processor 102 and the medicament injection unit 103 are located within one housing. In other potential embodiments, the processor 102 and medicament injection unit 103 are located within separate housings. These housings may be joined or separated from each other. The housing(s) may comprise any material suitable to location upon the skin of, away from, or implanted within a host 201. For example, the housing(s) may be comprised of stainless steel, ceramic, or any material suitable for placement upon the skin of, away from, or implanted within a host 201. The patch pump 101, as a single or multiple housing(s), may reside within the host 201, external to the host 201, upon the surface of the host 201, or a combination thereof, such as where the processor 102 housing resides upon the skin of the host 201 and the medicament injection unit 103 is implanted within the host 201. The patch pump 101 may be physically configured in any way and located in any place, though, to allow the medicament injection unit 103 to administer medicament.

The patch pump 101 may comprise any system for electrical power sourcing, such as a battery or external AC source. The power source may comprise any suitable source for powering the processor 102, medicament injection unit 103, and corresponding elements. In one exemplary embodiment, the power source may be a battery that is within the patch pump 101 housing capable of powering the processor 102 and medicament injection unit 103, such as a lithium ion battery. In other embodiments, the power source may be a different type of battery or may be an external power source such as a wall outlet. The power source may be any conventional electricity source capable of powering the processor 102, the medicament injection unit 103, and the like.

The processor 102 receives data and determines whether to output a medicament administration command to the medicament injection unit 103. The processor 102 may be any conventional central processing unit capable of receiving and processing data. The processor may comprise a memory unit or may be coupled to any suitable element that is a memory unit. In one exemplary embodiment, the processor is coupled to a memory unit so that electronic information may be stored and retained after the patch pump 101 is powered off.

The medicament injection unit 103, upon receiving a medicament administration command, is configured to administer a medicament to the host 201. The medicament injection unit 103 comprises a reservoir 301 that contains the medicament. The reservoir 301 may comprise any system for storing the medicament. In the present embodiment, the reservoir 301 is a single vessel capable of retaining the medicament, such as a glucagon. In other embodiments, the reservoir 301 may comprise a plurality of vessels, so that in the case where a medicament must be stored in separate vessels prior to mixture, the reservoir 301 may store the medicament properly for a set time. The reservoir 301 may be configured in any way to mix the medicament. For example, the reservoir 301 may comprise a motor, a breakaway wall, or the like, which, after receiving the medicament administration command, causes the parts of the medicament to be combined. The medicament injection unit 103 may further comprise a temperature protection system to stabilize the medicament during storage in the reservoir 301. For example, the temperature protection system may be a cooling system, an insulation system, a heating system, or the like. The medicament injection unit 103, however, may be configured in any suitable manner to store the medicament and prepare the medicament prior to administering the medicament to the host 201.

The medicament injection unit 103 may comprise any system for administering the medicament into the host 201. For example, in the present embodiment, the medicament injection unit 103 comprises an insertive delivery device and a motor system. The insertive delivery device comprises a needle and the motor lowers the needle into the host 201. The motor then pushes the medicament from the reservoir 301 through the needle and into the host 201. The insertive delivery device may comprise any system suitable for administering a medicament into a host 201. For example, the insertive delivery device may comprise a needle, cannula, or the like. The motor system may comprise a motor suitable for administering the medicament from the reservoir 301 into the host 201. For example, the motor, after receiving the medicament administration command from the processor 102, intramuscularly inserts a needle into the host 201 and then displaces the medicament in the reservoir 301 through the needle into the host 201. In other exemplary embodiments, the insertive delivery device may comprise a needle suitable for subcutaneous insertion. In other exemplary embodiments, the insertive delivery device may comprise a fixed device that may reside in an inserted fashion within the host 201. The motor system may comprise a plurality of motors suitable for administering the medicament from the reservoir 301 into the host 201. For example, a first motor may insert the insertive device into the host 201 and a second motor may push the medicament from the reservoir 301 through the insertive delivery device into the host 201. The medicament injection unit 103, however, may be configured in any suitable manner to administer medicament into the host 201 if the host 201 is experiencing changes in blood glucose concentration indicating a need for medicament.

The medicament injection unit 103 receives a medicament administration command from the processor 102. The processor 102 and medicament injection unit 103 are electrically coupled 403. The processor 102 and medicament injection unit 103 electrical coupling 403 may comprise any system that allows the medicament administration command to be sent by the processor 102 and received by the medicament injection unit 103. In the present embodiment, the processor 102 is electrically coupled to the medicament injection unit 103 by a wired connection. The processor 102 and medicament injection unit 103 electrical coupling 403, however, may comprise any suitable elements for transmitting an electrical signal, such as wireless communication.

The medicament administration command may comprise any form for the medicament injection 103 unit to configure to administer a medicament to the host 201. For example, in the present embodiment, the medicament administration command is a Universal Asynchronous Receiver-Transmitter communication protocol that initiates a motor to mechanically operate, resulting in the motor outputting motion while being powered from a separate source. In other exemplary embodiments, the medicament administration command may be a power signal to the motor itself. The medicament administration command may comprise a variety of signals distributed to various elements of the medicament injection unit 103, such as a motor and reservoir 301. In other embodiments, the medicament administration command may also comprise one signal transmitted to the medicament injection unit 103 and the medicament injection unit 103 distributes that command to its elements or acts upon that command. The medicament administration command, however, may be configured in any suitable manner to configure the medicament injection unit 103 to administer medicament to the host 201.

The processor 102 and a continuous glucose monitor 104 may be electrically coupled 401 for the processor 102 to receive continuous glucose sensor data indicative of the blood glucose concentration of the host 201. In the present embodiment, the continuous glucose monitor 104 resides within the housing of the patch pump 101. In other exemplary embodiments, the continuous glucose monitor 104 resides out of the housing of the patch pump 101. The continuous glucose monitor 104 may comprise any system for continuously monitoring blood glucose levels of the host 201. For example, the continuous glucose monitor 104 may be a Dexcom G6® sensor. The continuous glucose monitor 104 may, however, be any device suitable for providing the processor 102 with continuous glucose sensor data indicative of the blood glucose concentration of the host 201.

The processor 102 and continuous glucose monitor 104 are electrically coupled 401. The electrical coupling 401 may comprise any system for transmitting data between the continuous glucose monitor 104 and the processor 102. In the present embodiment, the continuous glucose monitor 104 is coupled to the processor 102 by a wire. In other embodiments, the continuous glucose monitor 104 may transmit the continuous glucose sensor data wirelessly to the processor 102. The electrical coupling 401 of the continuous glucose monitor 104 and processor 102, however, may comprise any system suitable for the continuous glucose monitor 104 to send the continuous glucose sensor data to the processor 102.

The processor 102 and a controller 105 may be electrically coupled 402 for the processor 102 to receive a first indicatory setting or second indicatory setting. In the present embodiment, the controller 105 is an external device outside of the housing of the patch pump 101 capable of sending a first or second indicatory setting to the processor 102. In other exemplary embodiments, the controller 105 may reside within the housing of the patch pump 101. The controller 105 may be any conventional device that allows indicatory settings to be sent to the processor 102.

The processor 102 and controller 105 are electrically coupled 402. The electrical coupling 402 may comprise any system for transmitting data between the controller 105 and the processor 102. In the present embodiment, the controller 105 is coupled to the processor 102 with a wire. In other embodiments, the continuous glucose monitor 104 may transmit the first indicatory setting or second indicatory setting wirelessly to the processor 102. The electrical coupling 402 of the controller 105 and processor 102, however, may comprise any system suitable for the controller 105 to send data to the processor 102.

The processor 102 and an electroencephalogram sensor 106 may be electrically coupled 404 for the processor 102 to receive electroencephalogram sensor data indicative of the electrical brain activity of the host 201. The electroencephalogram sensor 106 may comprise any conventional system that is able to output the electrical brain activity of the host 201 to the processor 102.

The processor 102 and electroencephalogram sensor 106 are electrically coupled 404. The electrical coupling 404 may comprise any system for transmitting data from the electroencephalogram sensor 106 to the processor 102. In the present embodiment, the electroencephalogram sensor 106 is coupled to the processor 102 with a wire. In other embodiments, the electroencephalogram sensor 106 may transmit the electroencephalogram sensor data wirelessly to the processor 102. The electrical coupling 404 of the electroencephalogram sensor 106 and processor 102, however, may comprise any system suitable for the electroencephalogram sensor 106 to send electroencephalogram sensor data indicative of the brain electrical brain activity of the host 201 to the processor 102.

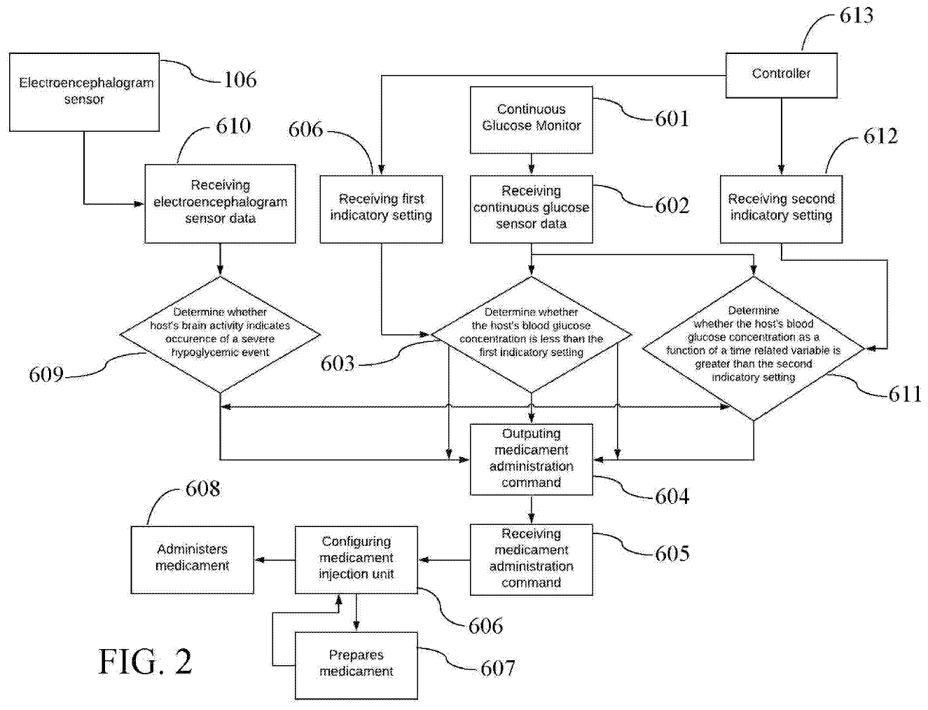

Referring now to FIGS. 1-2, in operation, the patch pump 101 determines whether the host 201 is experiencing changes in blood glucose concentration indicating a need for medicament and administers the medicament 608 to the host 201 if the host 201 is experiencing changes in blood glucose concentration indicating a need for medicament. The processor 102 first receives continuous glucose sensor data indicative of the blood glucose concentration of the host 201. The processor 102 may receive this information from any system that may provide continuous glucose sensor data of the host 201. In the present embodiment, the processor 102 receives this continuous glucose sensor data 602 from a continuous glucose monitor 104. The system providing the continuous glucose sensor data, however, may be any system capable of outputting continuous glucose sensor data indicative of the blood glucose concentration of the host 201.

The processor 102 must also receive a first indicatory setting 606. This first indicatory setting 603 sets a blood glucose concentration threshold so that the host 201 is considered to be experiencing changes in blood glucose concentration indicating a need for medicament when the blood glucose concentration of the host 201 drops below the first indicatory setting. The processor 102 may receive this first indicatory setting 606 from any system that may provide the first indicatory setting. In the present embodiment, the processor 102 receives the first indicatory setting 606 from the controller and the first indicatory setting is stored in the memory of the patch pump. This first indicatory setting may be rewritten if the processor 102 receives a new first indicatory setting from the controller. In other embodiments, the processor 102 may be hard-coded to use a permanent first indicatory setting. The first indicatory setting may be a non-rewritable entry within the memory of the patch pump. The processor 102, however, may receive the first indicatory setting regardless of the process and systems used for outputting the first indicatory setting.

While receiving the continuous glucose sensor data, the processor first determines 603 whether the host 201 may be experiencing changes in blood glucose concentration indicating a need for medicament by comparing the continuous glucose sensor data to the first indicatory setting. If the continuous glucose sensor data indicates the blood glucose concentration of the host 201 is less than the first indicatory setting, then the processor 102 will output the medicament administration command.

The processor 102 may also receive a second indicatory setting. This second indicatory setting sets a blood glucose concentration threshold as a function of a time related variable so that the host 201 may be considered to be experiencing changes in blood glucose concentration indicating a need for medicament when the blood glucose concentration rate of decrease of the host 201 is greater in magnitude than the second indicatory setting. The processor 102 may receive this second indicatory setting 612 from any system that may provide the second indicatory setting. In the present embodiment, the processor 102 receives the second indicatory setting 612 from the controller and the second indicatory setting is stored in the memory of the patch pump 101. This second indicatory setting may be rewritten if the processor 102 receives a new second indicatory setting from the controller. In other embodiments, the processor 102 may be hard-coded to use a permanent second indicatory setting. The second indicatory setting may be a non-rewritable entry within the memory of the patch pump. The processor 102, however, may receive the second indicatory setting 612 regardless of the process and systems used for outputting the second indicatory setting.

While receiving the continuous glucose sensor data, the processor may secondarily determine 611 whether the host 201 may be experiencing changes in blood glucose concentration indicating a need for medicament by comparing the continuous glucose sensor data to the second indicatory setting. If the continuous glucose sensor data indicates the blood glucose concentration as a function of time of the host 201 is decreasing at a greater rate (the magnitude is greater) than the second indicatory setting, then the processor 102 may output the medicament administration command 604. In the present embodiment, the processor 102 may output the medicament administration command if the processor 102 first determines 603 and secondarily determines 611 that the host 201 may be experiencing changes in blood glucose concentration indicating a need for medicament. The processor 102, however, need not secondarily determine 611 whether the host 201 may be experiencing changes in blood glucose concentration indicating a need for medicament to output the medicament administration command 604.

While receiving the continuous glucose sensor data 602, the processor 102 may thirdly determine whether the host 201 in part may be experiencing neurological characteristics of hypoglycemia by determining whether the electrical brain activity of the host 201 indicates the host 201 is experiencing neurological characteristics of hypoglycemia 609. The processor 102 may receive electroencephalogram sensor data indicative of the electrical brain activity of the host 201. The processor 102 may receive this information from any system that may provide electroencephalogram sensor data 610 of the host 201. In the present embodiment, the processor 102 receives this continuous electroencephalogram sensor data 610 from an electroencephalogram sensor 106. The system providing the electroencephalogram sensor data, however, may be any system capable of outputting electroencephalogram sensor data indicative of the electrical brain activity of the host 201.

If the electrical brain activity sensor data of the host 201 indicates the host 201 is experiencing neurological characteristics of hypoglycemia, then the processor 102 may output the medicament administration command 604. In the present embodiment, the processor 102 may output the medicament administration command 604 if the processor 102 determines whether the brain activity sensor data indicates the host 201 is experiencing neurological characteristics of a hypoglycemic event 609 and the first determination 603 indicates the magnitude of the blood glucose concentration of the host 201 is below the first indicatory setting. In one exemplary embodiment, the processor 102 may output the medicament administration command 604 if the processor 102 first determines 603 and thirdly determines 609 that the host 201 may be experiencing neurological characteristics of hypoglycemia. The processor 102 need not secondarily determine 611 whether the host 201 may be experiencing neurological characteristics of hypoglycemia to output the medicament administration command and the processor 102 also need not thirdly determine 609 whether the host 201 may be experiencing neurological characteristics of hypoglycemia to output the medicament administration command 604.

The processor 102 may determine that the electrical activity of the brain of the host 201 indicates the host 201 is experiencing neurological characteristics of a hypoglycemic event in any appropriate manner, such as analyzing the power and frequency of the beta (16-31 Hz), alpha (8-15 Hz), theta (4-7 Hz), and delta (<4 Hz) waves of the electroencephalography sensor data over a time related variable. In the present embodiment, the processor 102 may determine that the host 201 is experiencing neurological characteristics of hypoglycemia when there is an increase in the power of the delta band and theta band relative to the total power observed at the expense of the relative power in the alpha band. The processor 102 may also determine that the centroid frequency (the area under the curve of each frequency divided by 2) of theta increases and the centroid frequency of alpha decreases. In other embodiments, the processor 102 may determine that the host 201 is experiencing neurological characteristics of hypoglycemia when there is a decrease in centroid delta frequency and an increase or decrease in the absolute amplitude depending on the sleep stage of the host 201. In the present embodiment, the processor 102 comprises a programmed function that determines when there is an increase in the power of the delta band and theta band relative to the total power observed at the expense of the relative power in the alpha band to pre-set amplitude and frequency settings and further comprises a program function that determines when there is a decrease in centroid delta frequency and an increase or decrease in the absolute amplitude depending on the sleep stage of the host 201 to pre-set amplitude and frequency settings. The processor 102, however, may determine that the electrical activity of the brain of the host 201 indicates the host 201 is experiencing neurological characteristics of a hypoglycemic event in any appropriate manner.

The processor may be configured to receive amplitude, frequency, and state settings from the controller 104. In one exemplary embodiment, the processor 102 may receive pre-set amplitude and frequency settings from the controller 104 to determine when there is an increase in the power of the delta band and theta band relative to the total power observed at the expense of the relative power in the alpha band. In other exemplary embodiments, the processor 102 may receive pre-set amplitude and frequency settings from the controller 104 to determine when there is a decrease in centroid delta frequency and an increase or decrease in the absolute amplitude depending on the sleep stage of the host 201. In another exemplary embodiment, the processor 102 may further receive a state setting from the controller 104, where the state setting indicates whether the processor 102 should make the third determination 609 using an increase in the power of the delta band and theta band relative to the total power observed at the expense of the relative power in the alpha band, or make the third determination 609 using a decrease in centroid delta frequency and an increase or decrease in the absolute amplitude depending on the sleep stage of the host 201. The processor 102, however, may determine that the electrical activity of the brain of the host 201 indicates the host 201 is experiencing neurological characteristics of a hypoglycemic event in any appropriate manner.

After the processor 102 outputs the medicament administration command 604, the medicament injection unit 103 receives the medicament administration command 605 and begins configuring 606 itself to administer the medicament stored in the reservoir 301. During the configuration of medicament injection unit 606, the medicament may be prepared 607 in any appropriate manner for the medicament to treat changes in blood glucose concentration indicating a need for medicament. In the present embodiment, the reservoir 301 mixes two parts of the medicament in order for the medicament to be ready for treatment. In other exemplary embodiments, the medicament may not need to be mixed and the medicament need not be prepared. During the configuration of the medicament injection unit 606, the medicament injection unit 103 may also insert an administration system into the host 201. In the present embodiment, the medicament injection unit, may insert the insertive delivery device intramuscularly into the host 201. In other exemplary embodiments, the insertive delivery device may be inserted subcutaneously. The administration system may also reside in an inserted position within the host 201 and never be moved during the medicament injection unit configuration 606. The administration system may be any system and comprise any process needed to prepare the medicament injection unit to administer medicament to the host 201. The medicament injection unit may be configured in any process to prepare the medicament injection unit to administer medicament to the host 201.

After the medicament injection unit is configured 606, the medicament injection unit administers the medicament 608. The medicament injection unit may administer the medicament 608 in any appropriate manner based at least in part on a predetermined volume. For example, the medicament injection unit may push 1 mg of the medicament through the insertive delivery device into the host 201. In other exemplary embodiments, the medicament injection unit may push 0.5 mg into the host 201. The quantity and method of administration of the medicament 608, however, may be performed in any manner and amount appropriate for increasing the blood glucose concentration of the host 201.

I/We claim:

1) A hypoglycemia therapy device comprising:
a medicament injection unit comprising a reservoir, wherein the medicament injection unit is configured to administer a medicament stored in the reservoir into a host when receiving a medicament administration command; and
a processor configured to receive continuous glucose sensor data, the continuous glucose sensor data indicative of a host's blood glucose concentration, wherein the processor is configured to:
receive a first indicatory setting;
determine whether the host's blood glucose concentration is less than the first indicatory setting; and
output the medicament administration command, based at least in part on the host's blood glucose concentration being not greater than the first indicatory setting.

2) The device of claim 1, wherein the processor is further configured to:
receive a second indicatory setting;
determine whether the host's blood glucose concentration as a function of a time related variable is a greater rate of decrease than the second indicatory setting; and
output the medicament administration command, based at least in part on the host's blood glucose concentration as a function of the time related variable.

3) The device of claim 1, wherein the medicament injection unit, based at least in part on the medicament administration command, administers the medicament therapy into the host.

4) The device of claim 3, wherein the medicament injection unit prepares the medicament therapy prior to the medicament injection unit administering the medicament therapy into the host.

5) The device of claim 1, wherein the processor is in communication with a continuous glucose monitor, the processor configured to receive the continuous glucose sensor data from the continuous glucose monitor.

6) The device of claim 1, wherein the processor is in communication with a controller, the processor configured to receive the first indicatory setting from the controller.

7) The device of claim 2, wherein the processor is in communication with the controller, the processor configured to receive the second indicatory setting from the controller.

8) The device of claim 1, wherein the processor is configured to receive electroencephalogram sensor data, the electroencephalogram indicative of the host's electrical brain activity.

9) The device of claim 8 wherein the processor is further configured to:
determine whether the host's electrical brain activity indicates neurological characteristics of a hypoglycemic event; and
output the medicament administration command based at least in part on the neurological characteristics of the hypoglycemic event.

10) The device of claim 8, wherein the processor is in communication with an electroencephalogram sensor, the processor configured to receive the electroencephalogram sensor data from the electroencephalogram sensor.

11) A method of using a device to provide therapy for hypoglycemia, the method comprising:
configuring a medicament injection unit to administer a medicament stored in a reservoir that is within the medicament injection unit to a host after receiving a medicament administration command;
receiving continuous glucose sensor data by a processor, the continuous glucose sensor data indicative of a host's blood glucose concentration;
receiving a first indicatory setting by the processor;
determining by the processor whether the host's blood glucose concentration is less than the first indicatory setting; and
outputting by the processor the medicament administration command, based at least in part on the host's blood glucose concentration being less than the first indicatory setting.

12) The method of claim 11, further comprising:
receiving by the processor a second indicatory setting;
determining by the processor whether the host's blood glucose concentration as a function of a time related variable is a greater rate of decrease than the second indicatory setting; and
outputting by the processor the medicament administration command, based at least in part on the host's blood glucose concentration as a function of the time related variable.

13) The method of claim 11, wherein the medicament injection unit, based at least in part on the medicament administration command, administers the medicament therapy into the host.

14) The method of claim 13, wherein the medicament injection unit prepares the medicament therapy prior to the medicament injection unit administering the medicament therapy into the host.

15) The method of claim 11, wherein the continuous glucose sensor data is received by the processor from a continuous glucose monitor.

16) The method of claim 11, wherein the first indicatory setting is received by the processor from a controller.

17) The method of claim 12, wherein the second indicatory setting is received by the processor from the controller.

18) The method of claim 11, further comprising receiving electroencephalogram sensor data by the processor, the electroencephalogram indicative of a host's electrical brain activity.

19) The method of claim 18 further comprising:
determining by the processor whether the host's electrical brain activity indicates neurological characteristics of a hypoglycemic event; and
outputting by the processor the medicament administration command based at least in part on the neurological characteristics of the hypoglycemic event.

20) The method of claim 18, wherein the electroencephalogram sensor data is received by the processor from the electroencephalogram sensor.
